(19) United States
(12) Patent Application Publication
(10) Pub. No.: US 2023/0351592 A1
(43) Pub. Date: Nov. 2, 2023

(54) CLINICAL DECISION SUPPORT SYSTEM HAVING A MULTI-ORDERED HIERARCHY OF CLASSIFICATION MODULES

(71) Applicants: Frank Filiciotto, Bethlehem, PA (US); Dawei Liu, Sharon, MA (US)

(72) Inventors: Frank Filiciotto, Bethlehem, PA (US); Dawei Liu, Sharon, MA (US)

(21) Appl. No.: 18/307,350

(22) Filed: Apr. 26, 2023

Related U.S. Application Data
(60) Provisional application No. 63/363,654, filed on Apr. 27, 2022.
(60) Provisional application No. 63/387,140, filed on Dec. 13, 2022.

(51) Int. Cl. G06T 7/00; A61B 1/00

(52) U.S. Cl. CPC G06T 7/0012; A61B 1/00009; G06T 2207/20081

(57) ABSTRACT

A clinical support system and method for real-time activation of detection and characterization modules trained for identification or diagnosis of anomalies within a gastrointestinal tract are disclosed. A clinical support computer can be programmed to activate a first mucosa identification module and a first plurality of detection and characterization modules based at least in part on detection of connection to the endoscope, monitor an image stream from the endoscope using the first mucosa identification module to identify a mucosal tissue type, execute a first detection module from the first plurality of detection and characterization modules based on identifying the mucosal tissue type as a first mucosal tissue type, and process an image from the image stream received from the endoscope using the first detection module to identify a region of interest to output to the display device, wherein the region of interest identifies a potential anomaly within the image.

### PRIORITY CLAIM

This application claims the benefit of priority to U.S. Provisional Patent Application Ser. No. 63/363,654 filed, on Apr. 27, 2022 and U.S. Provisional Patent Application Ser. No. 63/387,140, filed on Dec. 13, 2022, the contents of which are incorporated herein by reference.

### BACKGROUND

Inspection of a patient's gastrointestinal (GI) tract using an endoscopic instrument is a fairly routine medical preventative/diagnostic medical procedure. For the upper GI tract, a doctor (or more generally a health care provider (HCP)) inspects the inside of a patient's esophagus, stomach, and duodenum (first portion of the small intestine). The instrument used in such a procedure is a thin lighted tube that includes a camera for viewing the internal surfaces of the organs that make up the upper GI tract—this instrument is commonly referred to as an endoscope. Accordingly, an upper endoscopy, also called an upper gastrointestinal endoscopy, is a procedure used to visually examine a patient's digestive system. Each section of the upper GI tract has distinctly different tissue, called mucosa, that can provide an indication of location within the GI tract to a well-trained eye. The visual examination performed within an endoscopy procedure is intended to identify anomalies that may require additional diagnosis.

Similarly, an endoscopic instrument can be used to visually examine a patient's lower gastrointestinal tract (lower GI tract). A lower GI endoscopy, also called colonoscopy or sigmoidoscopy, allows an HCP to view the mucosal lining of a patient's lower gastrointestinal tract. The procedure is typically used as a screening test in individuals with no symptoms, or to help diagnose unexplained abdominal pain, rectal bleeding, or a change in bowel habits. Routine colonoscopies are recommended after a certain age to enable early detection of cancer or other GI tract issues before they progress. Identification of anomalies within the GI tract requires a well-trained HCP and concentrated review of the camera image during the procedure. Endoscope manufacturers have developed some image processing technologies to assist in identifying potential anomalies, but these technologies can be difficult for the HCP to use in practice.

### OVERVIEW

Advances in Artificial Intelligence (AI) capabilities have generated an immense interest in exploring use cases in healthcare. From a practical standpoint, multiple different use cases may be applicable within individual healthcare settings. One such healthcare setting is an endoscopy suite in which an HCP may use an endoscope to inspect multiple discrete internal anatomical structures. The inspection of each discrete internal anatomical structure may correspond to one or more dedicated AI use cases (e.g., algorithms, trained AI models), and these dedicated AI use cases may be inapplicable to the other internal anatomical structures. Consider, for example, an Esophagogastroduodenoscopy (aka Upper GI endoscopy) during which an HCP may begin a procedure by inserting an endoscope into a patient's esophagus to examine the patient's esophageal mucosa before advancing the endoscope into the patient's stomach and finally the patient's duodenum. Due to the significant differences between the physiological structure of the mucosa within each of the esophagus, stomach, and duodenum, a trained AI model that reliably identifies and/or provides diagnosis for anomalies (e.g., cancerous lesions) within the one of these regions may be unable to identify and/or diagnose anomalies within the other regions which may be examined during a single procedure.

It is challenging to train HCPs on the continuously evolving nature of AI tools and, therefore, medical systems which require manual changes to intra-procedural settings to toggle between available AI tools creates a highly error prone environment. Furthermore, requiring manual changes of intra-procedural settings adds to the overall procedural time which reduces the number of cases an individual healthcare setting can support daily. There is a need for techniques for enhancing the quality and increasing the quantity of healthcare that is provided in individual healthcare settings in which multiple AI tools may be deployed.

This is an emerging technological area and existing systems are essentially stand-alone AI models that are trained to perform specific AI functions within discrete anatomical regions. These AI models can also be developed by multiple entities, which makes the AI capabilities more fragmented and challenging to be integrated.

To address these challenges the inventors have developed an end-to-end system which intelligently and seamlessly toggles between discrete AI models during a procedure based on the endoscope selection and signature of a tissue currently under observation.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example software architecture diagram in accordance with at least one example of this disclosure.

FIGS. 2A-2E illustrate an example intra-procedural process flow layered on the software architecture in accordance with at least one example of this disclosure.

FIG. 3A illustrates a machine learning model training diagram in accordance with at least one example of this disclosure.

FIG. 3B illustrates a machine learning model inference diagram in accordance with at least one example of this disclosure.

FIG. 4 illustrates a machine learning training technique in accordance with at least one example of this disclosure.

FIG. 5 illustrates a clinical decision support technique for automatic selection of detection and/or classification modules during a procedure in accordance with at least one example of this disclosure.

FIG. 6 illustrates a block diagram of an example machine upon which any one or more of the techniques discussed herein may perform in accordance with at least one example of this disclosure.

### DETAILED DESCRIPTION

In an example, a software architecture includes a multi-ordered hierarchy of modules in which the output of higher-level modules serves as a trigger to activate/deactivate lower-level modules. In this way, higher-level modules monitor current conditions of an operational environment and, based on these current conditions, selectively activate lower-level modules that are designed to operate in the current conditions. For example, a high-level module may determine a type of instrument that is connected to an endoscopy workstation and initialize (e.g., bring up to an operational state) a suite of lower-level modules that are designed for use with this instrument type (e.g., a colonoscope versus an esophagoscope) or even more granularly instrument model (e.g., esophagoscope A versus esophagoscope B). Additionally, or alternatively, a high-level module may determine a type of tissue that is currently being imaged by an endoscope and activate CADe and/or CADx modules that uniquely correspond to this tissue type. CADe/CADx modules are artificial intelligence computer modules for detection and/or classification of abnormal tissue. In this context, “CAD” is an acronym for Computer Aided Detection, with the CADe version indicating a version of computer aided detection configured to identify abnormal tissue. The CADx version refers to a computer aided detection system configured to identify and classify detected abnormal tissue. Additionally, in the context of this application the term “higher-level module(s)” refers to a software component that generates an output that is used to determine operational parameters corresponding to another software component, which is aptly referred to as a “lower-level module” relative to the aforementioned “higher-level module.” Additionally, the term “module” is used herein to define a functionally discrete piece of software code that is designed to perform a particular function or provide a particular service. A “module” is executable within a suitable computing system to produce defined outputs based on a defined input or set of inputs. In some examples, a “module” could encompass a complete application, such as an application executable within a clinical decision support system or a mobile computing device.

The software architecture discussed herein can also allow for an “app store” approach to CADe and CADx modules. The software architecture allows for an HCP to select from a variety of CADe/CADx modules that are compatible with the selected instruments to use in diagnosis within various portions of the GI tract. An interface can be provided to allow an HCP to associate a selected CADe/CADx module with different segments of the GI tract (which are detected during the procedure by a mucosal tissue identification module, as discussed in detail below).

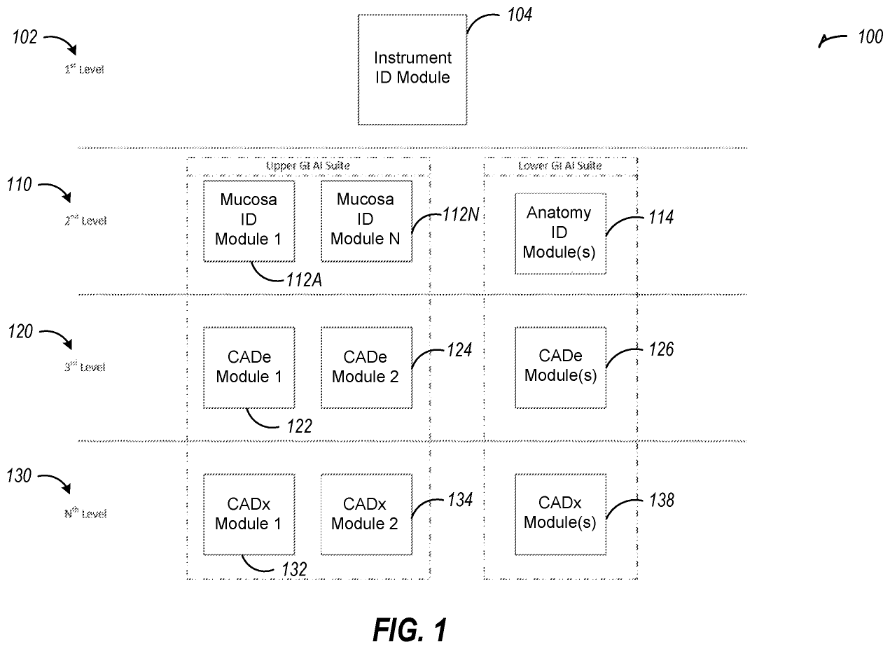

FIG. 1 illustrates an example software architecture diagram in accordance with at least one example of this disclosure. The example software architecture 100 includes multiple hierarchical levels, starting with a 1^{st} level 102 that includes an instrument identification module 104. The 2^{nd} level 110 in this example can include a plurality of mucosa identification modules 112A-112N including a first mucosa identification module 112A through an N^{th} mucosa identification module 112N—the disclosure may reference a mucosa identification module(s) 112 as shorthand for referencing one or more modules of the plurality of mucosa identification modules 112A-112N. Additionally, the plurality of mucosa identification modules 112A-112N may be referenced as the plurality of mucosa identification modules 112. In this example, the 2^{nd} Level 110 can also include one or more anatomy identification modules 114 for use within the lower GI tract. The anatomy identification modules 114 can operate based on mucosal tissue identification as well. As detailed below, both the mucosa identification module(s) 112 and the anatomy identification module(s) 114 can utilize trained machine learning models for identification of the respective tissue types. Further, in certain examples, the 2^{nd} level 110 can utilize a single mucosa identification module (e.g., mucosa identification module 112) that is programmed/trained to identify a variety of different mucosa tissue types. In other words, in certain examples, the plurality of mucosa identification modules 112A-112N are encapsulated into a single mucosa identification module.

In this example, the software architecture 100 can include a 3^{rd} level 120 that includes CADe modules for specific areas of the upper or lower GI tract. In this example, the 3^{rd} level 120 is illustrated as including esophageal CADe module 122 and stomach CADe module 124 as well as CADe module(s) 126 targeted for identification of anomalies within the lower GI tract. The software architecture 100 can include any number of levels, which is represented within the disclosure by the N^{th} level 130. The N^{th} level 130 is illustrated as including esophageal CADx module 132 and stomach CADx module 134 as well as CADx module(s) 138 targeted for characterization/classification of anomalies (e.g., characterization of polyps) within the lower GI tract. As indicated above, the various levels of software architecture 100 provides the structure to enable a clinical decision support system (CDSS) to seamlessly toggle between using multiple different AI tools within a single medical examination based on the current conditions (e.g., the current position of an endoscopic camera within a patient's GI tract as determined by a mucosa identification module).

The exemplary Endoscopy Software Architecture, software architecture 100, can include a first level including one or more instrument identification modules (e.g., instrument identification module 104) and one or more anatomy specific AI Suites. As illustrated below, the example Endoscopy Software Architecture includes an Upper GI AI Suite (e.g., Esophagogastroduodenoscopy (EGD) AI Suite) and a Lower GI AI Suite (e.g., Colonoscopy AI Suite). Individual ones of the AI Suites include a multitude of modules which are designed to perform specific AI functions through analyzing an image stream being captured by an endoscope. Furthermore, the output of higher-level modules within the AI Suites may serve as a determinative factor as to which lower-level modules operate at a given point in time. For example, a determination of a type and/or model of instrument that is selected by a user (or plugged into an endoscopy tower) may cause a CDSS to initialize a particular AI Suite that corresponds to the type and/or model of instrument. As another example, a most recent output of a 2^{nd} level module may dictate which 3^{rd} level modules are currently operational, and so on.

In the illustrated example, the 2^{nd} level 110 of the software architecture 100 includes various modules that are designed to determine a type of tissue that is currently being imaged by an endoscope. Specifically, within the Upper GI AI Suite resides mucosa identification module(s) (e.g., the plurality of mucosa identification modules 112) that are each designed to determine whether a particular type of mucosa is currently being imaged. Additionally, or alternatively, a single mucosa identification module (e.g., single AI-based tissue classification model) may be configured to determine which one of multiple different types of mucosae is currently being imaged. Although illustrated at the 2^{nd} level 110, these mucosa identification modules 112 (or more broadly anatomy identification modules if the target anatomy isn't mucosa) could be the first level in alternative implementations. In other words, the instrument identification within the illustrated 1^{st} level is an optional portion of the software architecture 100.

Furthermore, below the anatomy identification modules resides various AI models that are specifically developed and trained to analyze the type of tissue currently being viewed, as determined by the anatomy identification modules (which can also be AI models). For example, under circumstances in which the most recent image(s) analyzed at the 2^{nd} level 110 indicate that esophageal tissue/mucosa is being imaged by the endoscope, then at the 3^{rd} level 120 AI models are utilized which are configured to analyze esophageal tissue/mucosa (e.g., the esophageal CADe modules 122). Such AI models may include CADe and/or CADx type models, which in some implementations are separated into one or more levels as shown.

In a practical sense, this type of tiered software architecture enables an AI-enabled healthcare system to intelligently toggle between multiple different AI models, each of which are developed and trained for operation under specific operational circumstances, in a seamless and dynamic nature throughout a procedure as the operational conditions fluctuate. As a specific example, which is described in more detail below, the AI-enabled healthcare system may automatically transition from a first AI model configured to analyze a first anatomy type to a second AI model configured to analyze a second anatomy type as an endoscope passed from the first to second anatomy.

In this way, the concept adds significant value by creating an operational environment which, from the perspective of an end user (e.g., HCP), operates seamlessly to facilitate a singular goal (e.g., identifying, diagnosing, and ultimately treating diseased tissues) via dynamically toggling between various functionalities and/or AI tools responsive to changing operational conditions. At the same time, although appearing to be a singular tool from the perspective of an end user, the concept retains modularity from the developers' point of view which enables functionalities to be easily added, modified, and continuously improved as the underlying technology advances (e.g., without performing a complete software overhaul). Additionally, the concept facilitates end users to be provided with varying degrees of functionality in accordance with a subscription level (HCP A may subscribe to less or different functions than HCP B).

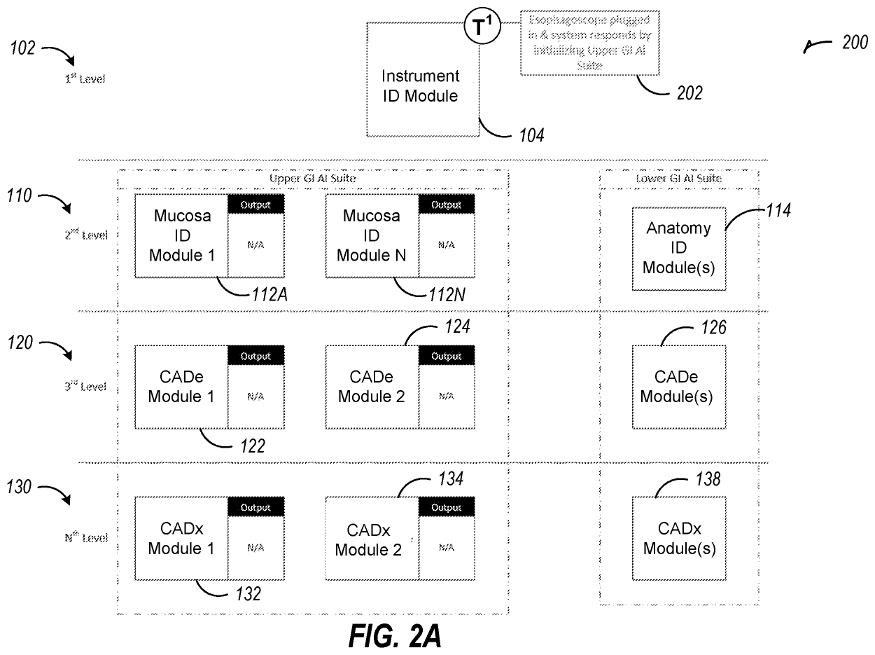

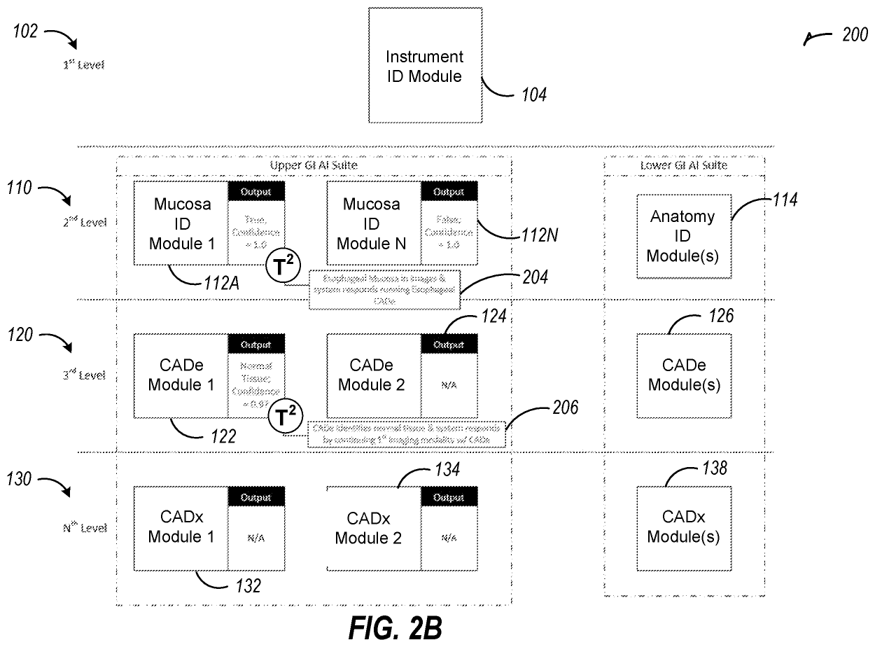

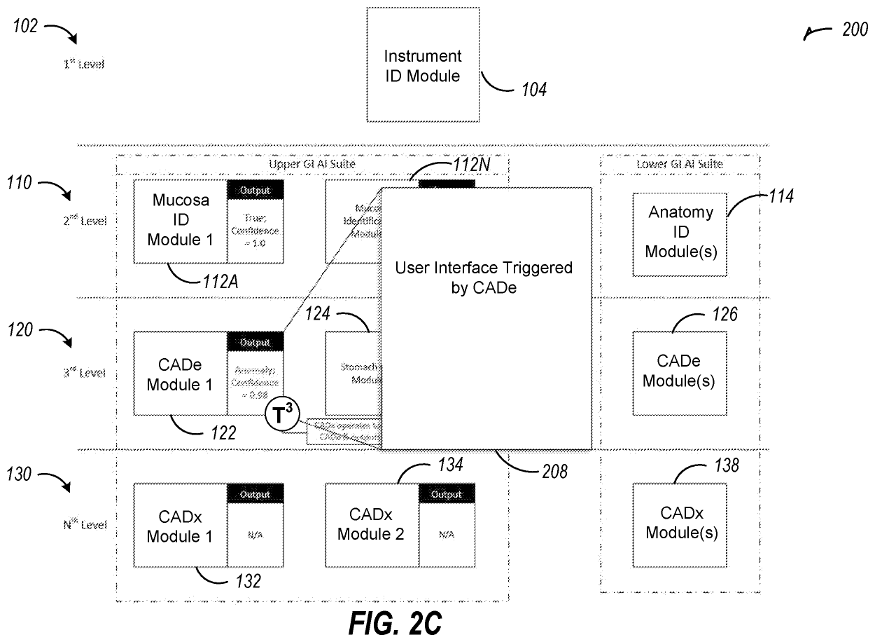

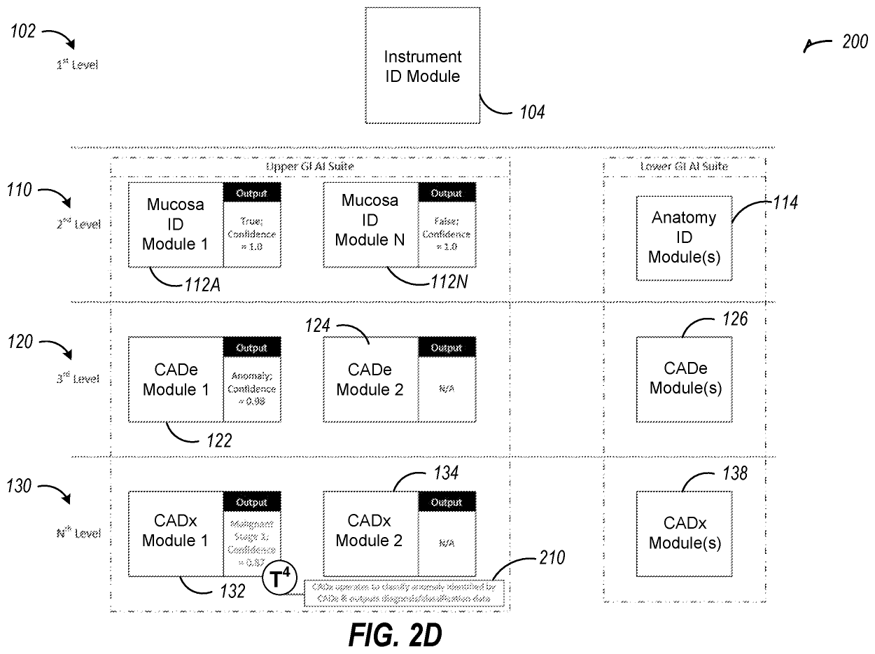

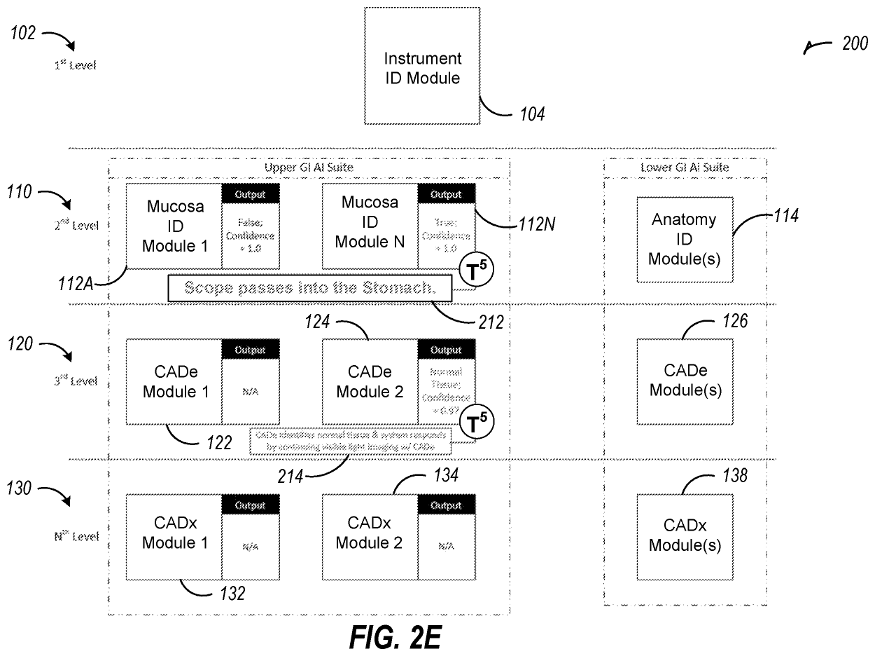

To further convey these concepts, an exemplary intra-procedural system flow is described below in relation to a sequence of figures. FIGS. 2A-2E illustrate an example intra-procedural process flow layered on the software architecture in accordance with at least one example of this disclosure. Each figure within FIGS. 2A-2E represents a different operation in the technique and is identified by different time stamps (T¹, T², etc.).

FIG. 2A—Time=T¹. In this example, the technique 200 can begin at operation 202, which occurs at a first moment in time T¹, and involves an HCP connecting an esophagoscope to the system operating the exemplary Endoscopy Software Architecture (e.g., software architecture 100). The instrument identification modules 104 recognizes that the instrument is an esophagoscope and responds by registering the intended anatomy, such as by initializing an Upper GI AI Suite (e.g., since the most likely use of an esophagoscope is an examination of the upper GI space). As illustrated below, because the instrument has just been connected and the examination is not yet underway (e.g., images of the upper GI space aren't yet being captured), the outputs of the various modules in the Upper GI AI Suite may be N/A (e.g., null) since images of the anatomies to be examined aren't yet being captured.

FIG. 2B—Time=T². At a second moment in time T², the technique 200 continues with operation 204 that involves the HCP inserting the esophagoscope into a patient's esophagus and the mucosa identification modules (e.g., the plurality of mucosa identification modules 112) operating to continuously (or periodically at an appropriate sample rate) determine the type of tissue currently being imaged. As illustrated, the mucosa identification modules 112 generate an output that indicates with a high degree of confidence (e.g., as measure by an appropriate threshold level such as 0.95 or greater—different confidence levels are within the scope of this disclosure) that esophageal mucosa is currently being imaged. In some examples, the system can generate a warning or other prompt to be displayed to the HCP if the type of tissue identified is determined not to be consistent with the registered anatomy. Note, the software can provide settings that allow the HCP to set a threshold confidence level for the mucosa identification modules 112. Based on this determination at the 2^{nd} level 110 of the software architecture 100, the technique 200 continues at operation 206 with the system executing an Esophageal CADe Module 122 to continuously analyze images being generated during the procedure. At time T², the output of the Esophageal CADe module 122 indicates that the tissue under observation is normal (e.g., non-anomalous). Therefore, the system continues with a 1^{st} imaging modality (e.g., visible light imaging) and continues to run the Esophageal CADe Module 122 concurrently while the mucosa identification modules 112 belonging to the Upper GI AI Suite also continuously run (or run at an appropriate sampling frequency). In this example, the plurality of mucosa identification modules 112A-112N continue to execute in the background in order to detect a change in the imaged mucosa tissue indicating a transition into a different section of the GI tract under examination.

In an alternative example (not illustrated), at time T², the HCP may activate an NBI module, in order to enable certain CADx modules to operate. In non-precancerous and depending on size and location within the colon, polypectomy can be performed to remove for follow-up assessment by pathologist or discarded.

FIG. 2C—Time=T³. At a third moment in time T³, the technique 200 can continue at operation 208 that includes the HCP manipulating the esophagoscope in such a way that abnormal tissue is being imaged. As a result, the Esophageal CADe Module 122 generates an output indicating that an anomaly exists within the space being imaged. In an example, a user interface dialog box can be output to a display device. Additionally, an audible sound (alert) can be generated to ensure the HCP is made aware of the detected abnormal tissue. For example, it should be appreciated that various CADe models are designed to detect anomalies such as polyps but not to characterize those polyps (e.g., as being benign, malignant, etc.). In some examples, graphics such as a region of interest bounding box is displayed overlaid on the endoscope image to alert the HCP of the detection of abnormal tissue.

Based on output from the Esophageal CADe Module 122, the system may respond by taking one or more predetermined actions. For example, the system may respond to the output from the Esophageal CADe Module 122 indicating an anomaly is detected by automatically activating functionalities to provide additional information regarding the abnormal tissue being imaged. In some embodiments, responsive to the output from the Esophageal CADe Module 122, the system may automatically deactivate a 1^{st} imaging modality (e.g., full spectrum or white light imaging) and activate a 2^{nd} imaging modality (e.g., Narrow Band Imaging (NBI) of blue or red light) in conjunction with an Esophageal CADx Module 132 (within the N^{th} level 130) that utilizes images captured via the 2^{nd} imaging modality to characterize anomalous esophageal tissues. In other examples, the Esophageal CADx Module 132 may be designed to operate utilizing white light, so the system does not activate the 2^{nd} imaging modality. In the immediately preceding example, the Esophageal CADe Module 122 may be aptly described as a “higher-level module” in relation to the Esophageal CADx Module 132 because the output of the Esophageal CADe Module 122 causes the system to trigger or activate functionality of the “lower-level” Esophageal CADx Module 132.

In some embodiments, the system may respond to the output of the Esophageal CADe Module 122 indicating that an anomaly is being imaged by generating a UI element (e.g., visual, audible, haptic) to prompt the HCP for an indication as to whether images captured via the 2^{nd} imaging modality are desired. If the HCP provides input indicating such 2^{nd} modality images are desired, the system may respond by switching from the 1^{st} imaging modality (e.g., visible light) to the 2^{nd} imaging modality (e.g., NBI). Then, responsive to images being captured via the 2^{nd} modality, the system may automatically utilize the Esophageal CADx Module 132 may analyze these images to generate an output indicating a tissue classification and corresponding confidence score. In certain examples, the HCP can manually turn on the NBI or another modality. In these examples, the information on new modality can be captured, without the need for detecting different types of images.

FIG. 2D—Time=T⁴. In this example, the technique 200 can continue at operation 210, which occurs at a fourth moment in time T⁴. Within operation 210, a 2^{nd} imaging modality is toggled on (either automatically or responsive to input from the HCP to do so). Responsive to the 2^{nd} imaging modality being activated, the system begins to provide the captured images to the Esophageal CADx Module 132 which classifies the anomalies identified via CADe (e.g., via the esophageal CADe module 122 executed in operation 206). For example, as illustrated, the Esophageal CADx Module 132 provides an output indicating that the anomaly is Malignant Stage 1, and that this classification corresponds to a 0.87 confidence.

FIG. 2E—Time=T⁵. In this example, the technique 200 can conclude with operations 212 and 214, at a fifth moment in time T⁵. Within these operations (212, 214) the HCP advances the esophagoscope all the way through the esophagus and into the stomach. As a result, the esophagoscope begins to capture images of the stomach mucosa. Since the mucosa identification modules (e.g., the plurality of mucosa identification modules 112A-112N) continue to run throughout the technique 200, the system seamlessly determines based on the outputs of these mucosa identification modules (112A-112N) to transition from operating the AI modules configured to analyze esophageal mucosa to AI modules configured to analyze stomach mucosa. Thus, as illustrated in FIG. 2E, the outputs of the Esophageal CADe module 122 becomes inapplicable (or null since it may cease to operate altogether) and the Stomach CADe Module 124 begins to output indications of whether the stomach mucosa currently being viewed is normal or abnormal. In practice, the technique 200 continues to operation during an entire endoscopic procedure with the discussion above illustrating a portion of such a procedure.

Alternative Use Case: Landmark Identification and Automatic Report Generation

During a colonoscopy, one important consideration is that a complete colonoscopy has been performed meaning that the HCP has advanced the colonoscope from the anus all the way to the end of the colon. The cecum is located at the end of the colon and marks a distinct transition from colonic space to the small intestine. Current best practices are for an HCP to advance the colonoscope all the way to the end of the colon and then to begin withdrawing the colonoscope while inspecting the colonic mucosa on the withdrawal.

The software architecture 100 can also be used to examine the colonic space and can auto populate aspects of a procedural report based on landmark identification. For example, an anatomy identification module, such as anatomy identification module 114, may be configured to analyze images to specifically identify the cecum. Once identified, the system can auto-populate a report with indications including:
a time when the cecum was reached
a duration that the colonoscope was being advanced
   This could be calculated by noting the time at which an anatomy identification module 1^{st} identified colonic mucosa (indicating the moment the colonoscope entered the anus) and noting time the cecum is identified, and then calculating advance time as the difference between these times.
   A duration that the colonoscope was being withdrawn (calculated as the reverse of the above—i.e., time from when cecum was identified minus time when colonic mucosa no longer identified in images when colonoscope is removed from anus)

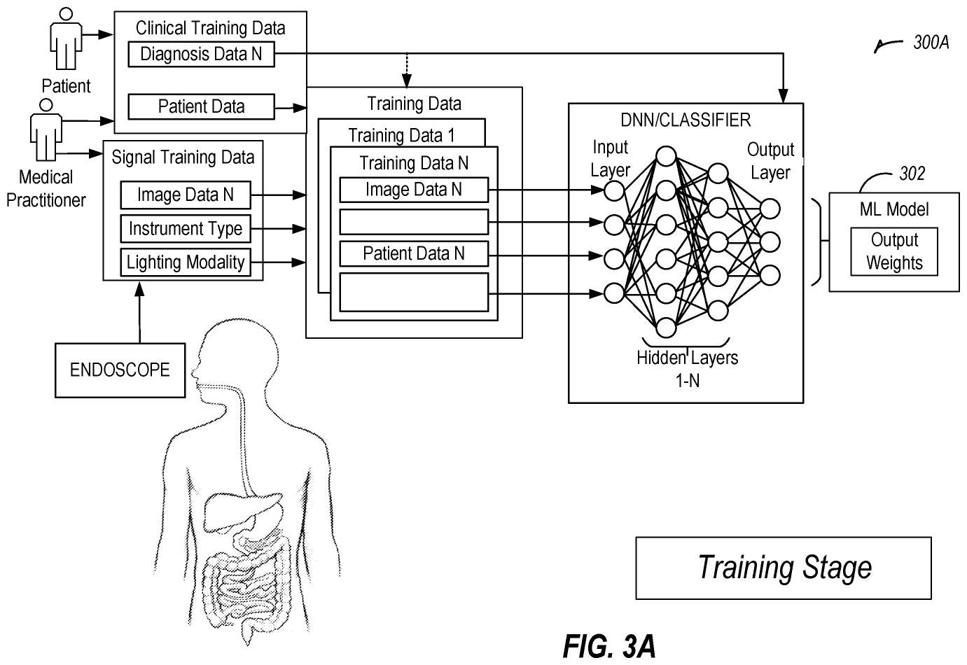

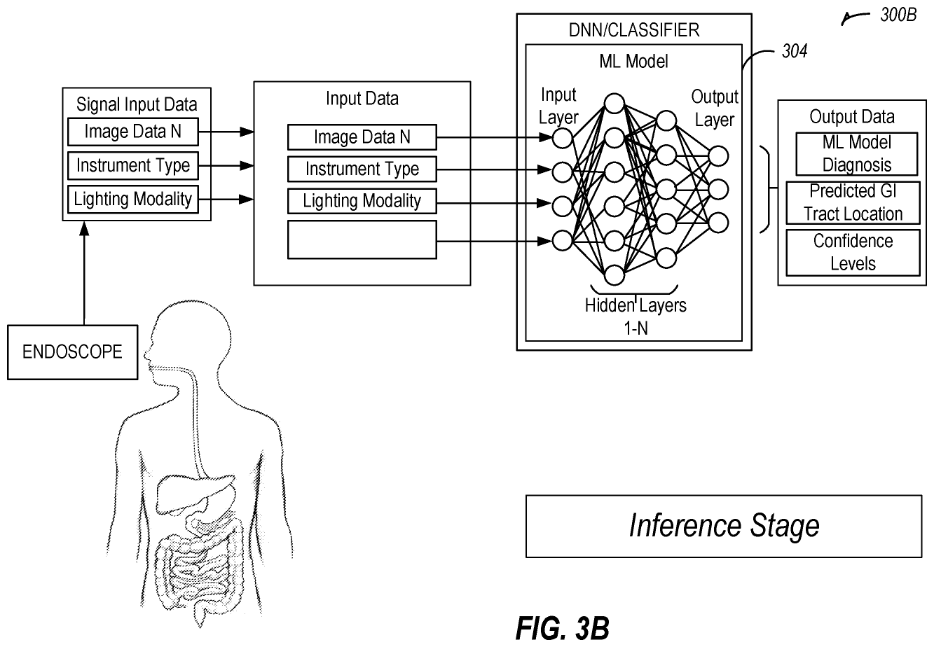

FIG. 3A illustrates a machine learning model training diagram 300A in accordance with at least one example of this disclosure. The diagram 300A illustrates components and inputs for training a model 302 using machine learning.

Machine Learning (ML) is an application that provides computer systems the ability to perform tasks, without explicitly being programmed, by making inferences based on patterns found in the analysis of data. Machine learning explores the study and construction of algorithms, also referred to herein as tools, that may learn from existing data and make predictions about new data. Although examples may be presented with respect to a few machine-learning tools, the principles presented herein may be applied to other machine-learning tools.

The machine-learning (ML) algorithms use data (e.g., action primitives and/or interaction primitives, goal vector, reward, etc.) to find correlations among identified features that affect the outcome. A feature is an individual measurable property of a phenomenon being observed. Example features for the model 302 may include diagnosis data (e.g., from a physician), reported patient outcome data, labeled mucosa tissue endoscope images, and corresponding endoscope location. The features, which may include and/or be called Clinical Data, may be compared to input data, such as endoscopic tissue images.

The concept of a feature is related to that of an explanatory variable used in statistical techniques such as linear regression. Choosing informative, discriminating, and independent features is important for effective operation of ML in pattern recognition, classification, and regression. Features may be of different types, such as numeric features, strings, and graphs.

During training, a ML algorithm analyzes the input data based on identified features and optionally configuration parameters defined for the training (e.g., environmental data, state data, patient data such as demographics and/or comorbidities, etc.). The result of the training is the model 302, which is capable of taking inputs to produce a complex task. In this example, the model 302 will be trained to identify different types of mucosa from input endoscope images.

In an example, input data may be labeled (e.g., for use as features in a training stage). Labeling may include identifying mucosa tissue and location within the GI tract the tissue was located. Labeled training images may be weighted, and/or may be used to generate different versions of the model 302.

Input training data for the model 302 may include Clinical Data that can include patient data, such as weight, height, and any other patient data that might impact risk factors associated with targeted diagnosis.

A neural network, sometimes referred to as an artificial neural network, is a computing system based on consideration of biological neural networks of animal brains. Such systems progressively improve performance, which is referred to as learning, to perform tasks, typically without task-specific programming. For example, in image recognition, a neural network may be taught to identify images that contain an object by analyzing example images that have been tagged with a name for the object, and having learned the object and name, may use the analytic results to identify and/or classify the object in untagged images. In FIG. 3A for example, the model 302 may be trained to identify mucosa tissue type and/or location within the GI tract, and/or with a percentage likelihood and/or confidence of tissue location.

A neural network is based on a collection of connected units called neurons, where each connection, called a synapse, between neurons can transmit a unidirectional signal with an activating strength that varies with the strength of the connection. The receiving neuron can activate and propagate a signal to downstream neurons connected to it, typically based on whether the combined incoming signals, which are from potentially many transmitting neurons, are of sufficient strength, where strength is a parameter.

A deep neural network (DNN) is a stacked neural network, which is composed of multiple layers. The layers are composed of nodes, which are locations where computation occurs, loosely patterned on a neuron in the human brain, which fires when it encounters sufficient stimuli. A node combines input from the data with a set of coefficients, and/or weights, that either amplify or dampen that input, which assigns significance to inputs for the task the algorithm is trying to learn. These input-weight products are summed, and the sum is passed through what is called an activation function for a node, to determine whether and to what extent that signal progresses further through the network to affect the ultimate outcome. A DNN uses a cascade of many layers of non-linear processing units for feature extraction and transformation. Each successive layer uses the output from the previous layer as input. Higher-level features are derived from lower-level features to form a hierarchical representation. The layers following the input layer may be convolution layers that produce feature maps that are filtering results of the inputs and are used by the next convolution layer.

The DNN may be a specific type of DNN, such as a convolutional neural network (CNN), a recurrent neural network (RNN), a Long Short Term Memory (LSTM), or the like. Other artificial neural networks may be used in some examples. A classifier may be used instead of a neural network in some examples. A classifier may not include hidden layers, but may classify a particular input as corresponding to a particular output. For example, for a mucosa image input data, an identification of location within the GI tract may be generated by the classifier.

The input data for training the model 302 may include data captured from an endoscope, with labeled data from a medical practitioner. The model 302 may be used in an inference stage (described in further detail below with respect to FIG. 3B) for determining location within the GI tract of a particular input mucosa tissue image.

As shown in FIG. 3A, training data may include signal training data that is comprised of endoscopic images of mucosa tissue as well as additional procedure related data such as endoscope instrument type and lighting modality. In some examples, the signal training data includes annotation or labeling data that is provided by a medical practitioner (e.g., as image labeling data). For example, a medical practitioner may annotate each input endoscopic mucosa tissue image (e.g., each Image Data N). In some examples, the patient and/or medical practitioner can also provide clinical data that is used to train the model 302. Clinical data can include diagnosis data and patient data.

Based on the signal training data and/or annotation training data, the model 302 may generate output weights corresponding to individual processing nodes that are spread across an input later, an output layer, and one or more hidden layers. The model 302 and trained weights may later be used to infer an indication of GI tract location based on endoscopic input images of mucosa tissue.

FIG. 3B illustrates a machine learning model inference diagram 300B in accordance with at least one example of this disclosure. In the inference diagram 300B, a model 304 (e.g., the model 302 after training, and/or as updated, etc.) may be used to output a prediction, such as a location within the GI tract or mucosal tissue type, or the like. A confidence level and/or weighting may be output as the prediction, or in addition to other predictions discussed above. The machine learning model inference diagram 300B may represent an exemplary computer-based clinical decision support system (CDSS) that is configured to assist in predicting a position of an endoscopic instrument within a patient's GI tract.

As shown in FIG. 3, the model 304 may receive signals from an endoscope as input, such as image data of a patient's GI tract. The input data can also include endoscopic instrument type and lighting modality, among other things. The model 304 may generate an output (e.g., an inference) that includes a location within the GI tract or a mucosal tissue type, or the like. The model 304 may have a run-time that occurs while a patient is undergoing an endoscopic procedure. The model 304 may provide a physician with real-time or near real-time information about a location within the GI tract. The GI tract location information may also be used by the CDSS to activate or deactivate certain other AI/ML modules that assist with detection and/or characterization of tissue abnormalities (e.g., CADe and/or CADx modules).

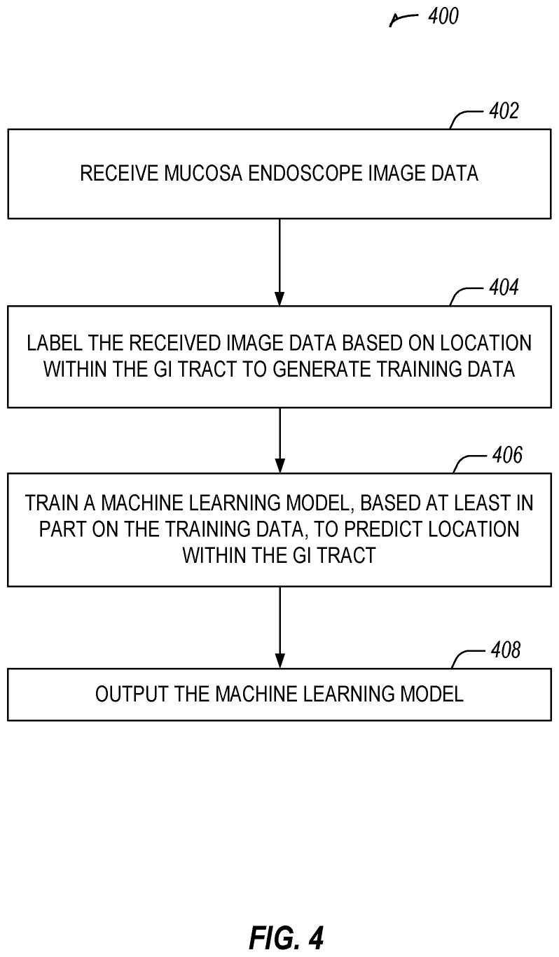

FIG. 4 illustrates a machine learning training technique 400 in accordance with at least one example of this disclosure. In this example, the technique 400 outlines the basic steps for training a machine learning model to detect different mucosal tissue types and/or GI tract locations based on input endoscopic images. The technique 400 can include operations such as receiving mucosa endoscope image data at 402, labeling the image data at 404, training a machine learning model at 406, and outputting the machine learning model at 408.

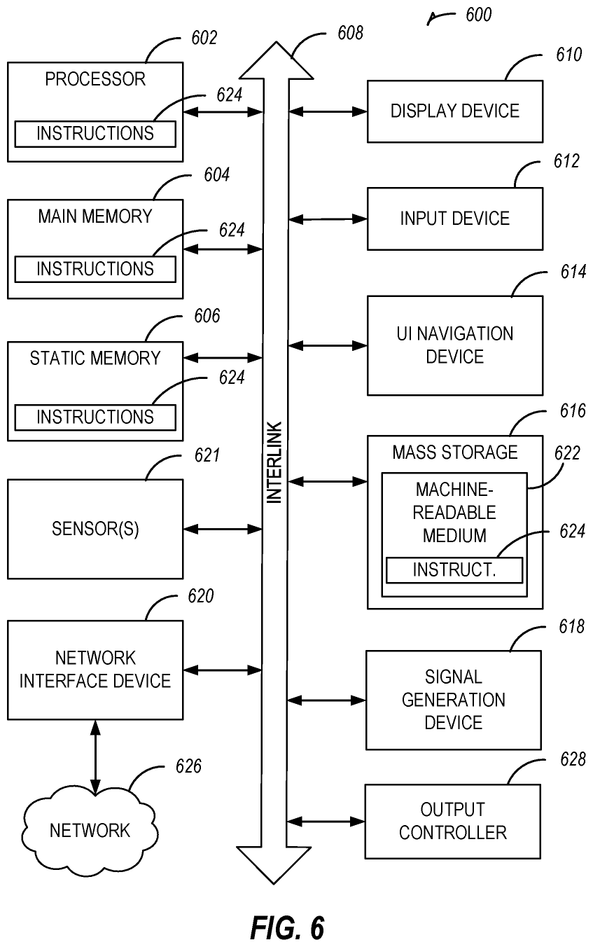

In this example, the technique 400 can begin at 402 with a system (as the computer system detailed in FIG. 6 below) receiving a plurality of mucosal tissue images from an endoscope. The mucosal tissue images can originate from the upper GI tract and/or the lower GI tract. Images from the upper GI tract can include images of mucosa including esophageal mucosa, gastric mucosa, and duodenal mucosa, which each have visually distinct aspects. The image data from the lower GI tract can include images from the cecum, the ascending colon, the right the colic flexure, the transverse colon, the left colic flexure, the descending colon, the sigmoid colon, the rectum, and the anal canal, among other distinct areas of the lower GI tract.

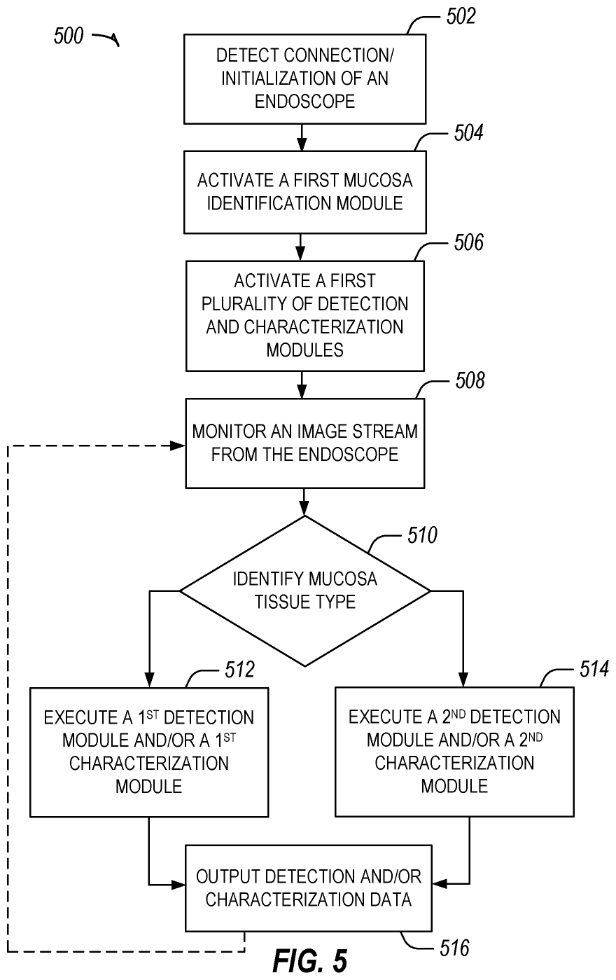

The technique 400 can continue at 404 with the system receiving label data for each of the training mucosa endoscopic images received in operation 402. The labeling operation at 404 may also include weighting the training images according to strength of representation of a particular mucosal tissue type. The output of operation 404 can include a set of training data that includes the annotations associated with each image of the received image data. At 406, the technique 400 can continue with the training data being used to train a machine learning model that can subsequently be used to predict a location within the GI tract of an input endoscopic image. FIG. 3A above discusses additional details on training a machine learning model that can be implemented within the context of technique 400. Once trained, the technique 400 can conclude at operation 408 with the system outputting the machine learning model for use in predicting GI tract location and/or mucosal tissue type. In an example, the trained machine learning model output by technique 400 can be used within technique 500 discussed below in reference to FIG. 5.

FIG. 5 illustrates a clinical decision support technique 500 for automatic selection of detection and/or classification modules during a procedure in accordance with at least one example of this disclosure. In this example, the technique 500 can include operations such as: detecting an endoscope at 502, activating a mucosa identification module at 504, activating a first plurality of detecting and characterization modules at 506, monitoring an image stream at 508, identifying a mucosal tissue type at 510, executing a 1^{st} detection and/or characterization module at 512, alternatively executing a 2^{nd} detection and/or characterization module at 514, and outputting detecting and/or characterization data at 516. The technique 500 discusses detect and characterization modules, which can be CADe and CADx module respectively.

In this example, the technique 500 can optionally begin at 502 with the system (e.g., a CDSS) detecting an endoscope type. For example, the system may detect a connection with an endoscope and determine that the endoscope belongs to a “esophagoscope” category of instruments. Additionally, or alternatively, the system may more granularly determine the specific model of the detected endoscope (e.g., the system may identify the endoscope as the GIF-XP190N version of the EVIS EXERA III sold by Olympus Corporation of the Americas). In this operation detection of the endoscope type can be triggered by connection of the instrument to the CDSS. In other example, detecting the endoscope type can be triggered by initialization of the instrument after connection with a CDSS, or some other logical point in the procedure prior to use of the instrument.

At 504, the technique 500 continues with the system activating a first mucosa identification module. Activation of the mucosa identification module can be based on the type and/or model of endoscopic instrument connected to the system. For example, based on the system having determined at 502 that an instrument belonging to the “esophagoscope” category of instruments is connected, the system may start running a mucosa identification module that is designed to utilize one or more classification type ML models to analyze endoscopic images and output an indication of whether the images depict squamous mucosa, gastric mucosa, or villous mucosa. As discussed above, different types of endoscopes are used for different procedures, such as an upper GI tract procedure versus a lower GI tract procedure. Correspondingly, mucosa identification modules can be tailored to the upper GI tract versus the lower GI tract (or based on other specific procedural traits). In certain examples, a mucosal tissue identification module can be trained to span the entire GI tract.

At 506, the technique 500 continues with activation of a first plurality of detection and characterization modules. In this example, activation of the detection and characterization modules is also based on the type of endoscopic instrument detected. For example, responsive to the determination of the type (e.g., esophagoscope) and/or model (e.g., GIF-XP190N) of the endoscope, the system may begin running the Upper GI AI Suite depicted in FIG. 1. Accordingly, it will be appreciated that aspects of each of operations 504 and 506 may be performed based on the outcome and/or determination of operation 502. In another example, operations 504 and 506 may be based on another system input indicating where within a patient's GI tract the procedure is being performed. Another factor in determining which mucosa identification module to activate at 504 and which plurality of detecting and characterization modules to activate at 506 can involve available light modes within the connected instrument (e.g., normal white light versus NBI, or other lighting types).

At 508, the technique 500 can continue with the system monitoring an image stream generated by the endoscope. Operation 508 can operate continually throughout a procedure using the endoscopic instrument. At 510, the technique 500 can continue with the system using the active mucosa identification module (e.g., the first mucosa identification module) to continually (or periodically at a suitable sample rate) identify a mucosal tissue type within individual images processed from the image stream monitored in operation 508. Operation 510 can run on every image within the monitored image stream, or it can be executed periodically on an image extracted from the monitored image stream. In this example, when a first mucosal tissue type is identified at 510, the technique 500 can continue at operation 512 with the system executing a first detection module and/or a first characterization module. For example, based on operation 510 resulting in an indication that squamous mucosa is being imaged currently, the system may perform operation 512 in which the Esophageal CADe module 122 and/or Esophageal CADx module 132 may be executed since squamous mucosa lines most of the esophagus. Executing a detection or characterization module at operation 512 can include selecting a lighting modality supported by the endoscopic instrument in use. At 516, the technique 500 continues with the system outputting detection and/or characterization data to assist the HCP in performing the ongoing procedure. Outputting detection data at 516 can include outputting a region of interest overlaid on an endoscopic image to identify abnormal tissue to an HCP. Operation 516 can also include displaying a user prompt to provide analysis options, such as switching lighting modalities and/or executing a characterization module.

In certain examples, the technique 500 loops through operations 508-516 in real-time or near real-time to continually update graphical display of detection and/or characterization data on a display device displaying the endoscopic image stream. In an example, after the first detection module generates a region of interest identifying potentially abnormal tissue, the technique 500 can loop back to operation 512 to execute a first characterization module. The first characterization module can then output a tissue classification at operation 516. In certain examples, the characterization module can also output a confidence score or indicator alone with the tissue classification.

Back at operation 510 (which continually or periodically executes contemporaneously with operations 512 and 516 as described above), when the mucosa identification module identifies a second mucosal tissue type, the technique 500 can continue at operation 514 with the system executing a second detection module and/or second characterization module. For example, based on operation 510 resulting in an indication that gastric mucosa is being imaged currently, the system may automatically toggle to performing operation 514 in which the Stomach CADe module 124 and/or Stomach CADx module 134 may be executed since gastric mucosa lines the stomach. The technique 500 can conclude at operation 516 with the system outputting detection and/or characterization data to assist the HCP in performing the ongoing procedure. In this example, the detection of a second mucosa tissue type at operation 510 is an indication that the procedure has transitioned into a different area of the GI tract. In an example, upon detection of the procedure transitioning to a new area of the GI tract and indication of this event can be output to a display device coupled to the system to further assist the HCP.

The above invention is discussed in reference to an endoscope application, but the same concept can be applied to endoscopic, ultrasound, and microscope diagnostic applications. The system described herein can transition between AI modules intra-procedurally and will attach metadata that identifies which AI modules is operating for different portions of a video stream. The system supports modular insertion of AI modules as functionality is added or updated. The system can generate reports detailing which algorithms (modules) where selected and why throughout a procedure.

FIG. 6 illustrates a block diagram of an example machine 600 upon which any one or more of the techniques (processes) discussed herein may perform in accordance with some embodiments. In alternative embodiments, the machine 600 may operate as a standalone device and/or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NFC), etc.) connection to communicate and/or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600, and includes digital or analog communications signals or other intangible medium to facilitate communication of such software.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

### EXAMPLES

The following, non-limiting examples, detail certain aspects of the present subject matter to solve the challenges and provide the benefits discussed herein, among others.

Example 1 is a gastrointestinal (GI) tract mucosa identification system for training a machine learning model for use in a computer-based clinical decision support system to assist in real-time selection of computer aided detection and characterization modules trained for specialized anatomical regions. In this example, the GI tract mucosa identification system can include a data of mucosa images and a computing system with processing circuitry and a memory device. The memory device can include instructions, which when executed by the processing circuitry, cause the processing circuitry to perform operations including labeling, training a machine learning model and storing the machine learning model. The labeling can include labeling a plurality of mucosa images from the database based on location within the GI tract to generate training data. The training the machine learning model can including using the training data to predict location of an endoscope within a GI tract based on real-time mucosal imaging from the endoscope.

Example 2 is a method for training a machine learning model for use in a computer-based clinical decision support system providing real-time selection of computer aided detection and characterization modules trained for specialized identification or diagnosis of anomalies within specific anatomical regions. In this example, the method can include operations for receiving data, labeling the received data, training the machine learning model, and outputting the machine learning model. The receiving data can including data captured by an endoscope that includes mucosa images from various portions of a GI tract. The labeling the received data can including labeling based on location within the GI tract to generate training data. Training the machine learning model can be done using the training data to training a model to predict location of an endoscope within a GI tract based on real-time mucosal imaging from the endoscope.

In Example 3, the subject matter of Example 2 can optionally include receiving the data by receiving endoscope images including esophageal mucosa, gastric mucosa, duodenal mucosa.

In Example 4, the subject matter of any one of Examples 2 and 3 can optionally include the training the machine learning model by training an upper GI tract model and a lower GI tract model.

In Example 5, the subject matter of Example 4 can optionally include the training the lower GI tract model by using training data specific to the lower GI tract.

In Example 6, the subject matter of Example 5 can optionally include the training data specific to the lower GI tract includes labeled endoscopic images from a group of images selected from one or more of the following anatomical areas: cecum, ascending colon, right colic flexure, transverse colon, left colic flexure, descending colon, sigmoid colon, rectum, and anal canal.

Example 7 is an endoscopic clinical support system for real-time activation of computer aided detection and characterization modules trained for specialized identification or diagnosis of anomalies within a gastrointestinal (GI) tract of a patient. In this example, the system can include an endoscope and a clinical support computing device. The endoscope can include a sensor and a lighting component configured to generate medical image data. The clinical support computing device is communicatively coupled to the endoscope. The clinical support computing device also includes processing circuitry, a display device configured to display the medical image data and related user interface graphics, and a memory, including instructions, which when executed by the processing circuitry, cause the processing circuitry to perform operations including the following. Activating a first mucosa identification module and a first plurality of detection and characterization modules based at least in part on detection of connection to the endoscope. Monitoring an image stream from the endoscope using the first mucosa identification module to identify a mucosal tissue type. Executing a first detection module from the first plurality of detection and characterization modules based on identifying the mucosal tissue type as a first mucosal tissue type. As well as processing an image from the image stream received from the endoscope using the first detection module to identify a region of interest to output to the display device, wherein the region of interest identifies a potential anomaly within the image.

In Example 8, the subject matter of Example 7 can optionally include activating the first mucosa identification module by receiving endoscope type or model information identifying the endoscope.

In Example 9, the subject matter of Example 8 can optionally include receiving endoscope type or model information including receiving lighting information identifying the different lighting types the lighting component is capable of generating.

In Example 10, the subject matter of any one of Examples 7 to 9 can optionally include the memory further including instructions, which when executed by the processing circuitry, cause the processing circuitry to perform operations such as: continually monitoring images extracted from the image stream received from the endoscope using the first mucosa identification module; and executing a second detection module from the first plurality of detection and characterization modules based on the first mucosa identification module identifying the mucosal tissue type as a second mucosal tissue type.

In Example 11, the subject matter of Example 10 can optionally include executing the second detection module by outputting an indication to the display device that the endoscope has begun imaging a different region of the GI tract.

In Example 12, the subject matter of any one of Examples 7 to 11 can optionally include the monitoring the image stream from the endoscope using the first mucosa identification module can include implementing a machine learning model, trained based at least in part on training data including a plurality of labeled mucosa images from various locations within a GI tract, to predict location of the endoscope within a GI tract based on real-time mucosal imaging from the endoscope.

In Example 13, the subject matter of any one of Examples 7 to 12 can optionally include the monitoring the image stream using the first mucosa identification module can include periodically processing an image extracted from the image stream using the first mucosa identification module.

In Example 14, the subject matter of any one of Examples 7 to 13 can optionally include the executing the first detection module can include selecting a first imaging modality from a plurality of imaging modalities supported by the endoscope.

In Example 15, the subject matter of Example 14 can optionally include identifying the region of interest by selecting a second imaging modality from the plurality of imaging modalities supported by the endoscope.

In Example 16, the subject matter of Example 15 can optionally include the memory further including instructions, that when executed by the processing circuitry, cause the processing circuitry to perform operations comprising executing a first characterization module from the plurality of detecting and characterization modules based at least in part on identifying the region of interest.

In Example 17, the subject matter of Example 16 can optionally include executing the first characterization module by outputting an indication of a tissue classification and corresponding confidence score related to the region of interest to the display device.

In Example 18, the subject matter of any one of Examples 14 to 17 can optionally include identifying the region of interest including displaying a user prompt on the display device providing an option to activate a second imaging modality from the plurality of imaging modalities supported by the endoscope.

Example 19 is a system for real-time activation of computer aided detection and characterization modules trained for specialized identification or diagnosis of anomalies within a gastrointestinal (GI) tract of a patient. The system can include one or more processing units and a computer-readable medium having encoded thereon computer-executable instructions to cause the one or more processing units to perform operations such as the following. Receive, during a medical examination, image data that is generated by a medical imaging device. Provide, at a first time during the medical examination, a first portion of the image data to one or more anatomy identification modules that is configured to output individual indications of whether individual portions of the image data depict a first anatomy type or a second anatomy type. Receive, from the one or more anatomy identification modules, a first indication that the first portion of the image data depicts the first anatomy type. Responsive to the first indication, deploying a first CAD model to analyze the first portion of the image data, wherein the first CAD model is configured to generate annotations in association with anomalies depicted in images of the first anatomy type. Provide, at a second time during the medical examination that is subsequent to the first time, a second portion of the image data to the one or more anatomy identification modules. Receive, from the one or more anatomy identification modules, a second indication that the second portion of the image data depicts the second anatomy type. As well as, responsive to the second indication, deploying a second CAD model to analyze the second portion of the image data, wherein the second CAD model is configured to generate annotations in association with anomalies depicted in images of the second anatomy type.

In Example 20, the subject matter of Example 19 can optionally include the computer-executable instructions further cause the one or more processing units to: receive an output from the first CAD model that indicates a detection of an anomaly within the first portion of the image data; and responsive to the output from the first CAD model, deploying a third CAD model to analyze at least some of the first portion of the image data, wherein the third CAD model is configured to generate an output that classifies the anomaly detected by the first CAD model.

In Example 21, the subject matter of Example 20 can optionally include deploying the third CAD model by activating an alternative lighting modality on the medical imaging device.

In Example 22, the subject matter of Example 21 can optionally include the alternative lighting modality being a narrow band imaging modality of blue or red light.

In Example 23, the subject matter of any one of Examples 20 to 22 can optionally include the third CAD model being configured to output a tissue classification and confidence score related to the tissue classification.

In Example 24, the subject matter of any one of Examples 19 to 23 can optionally include the first anatomy type being squamous mucosa corresponding to an esophageal anatomical region and the second anatomy type is gastric mucosa corresponding to a stomach anatomical region.

In Example 25, the subject matter of any one of Examples 19 to 24 can optionally include the image data including a sequence of multiple image frames that are generated during the medical examination by an endoscope.

In Example 26, the subject matter of any one of Examples 19 to 25 can optionally include deploying the first CAD module by outputting an indication of the first anatomy type.

In Example 27, the subject matter of any one of Examples 19 to 26 can optionally include deploying the second CAD model includes outputting an indication of the second anatomy type.

What is claimed is:

1. An endoscopic clinical support system for real-time activation of computer aided detection and characterization modules trained for specialized identification or diagnosis of anomalies within a gastrointestinal (GI) tract of a patient, the system comprising:
an endoscope including a sensor and a lighting component configured to generate medical image data;
a clinical support computing device communicatively coupled to the endoscope, the clinical support computing device including:
processing circuitry;
a display device configured to display the medical image data and related user interface graphics; and
memory, including instructions, which when executed by the processing circuitry, cause the processing circuitry to perform operations comprising:
activating a first mucosa identification module and a first plurality of detection and characterization modules based at least in part on detection of connection to the endoscope;
monitoring an image stream from the endoscope using the first mucosa identification module to identify a mucosal tissue type; and
executing a first detection module from the first plurality of detection and characterization modules based on identifying the mucosal tissue type as a first mucosal tissue type; and
processing an image from the image stream received from the endoscope using the first detection module to identify a region of interest to output to the display device, wherein the region of interest identifies a potential anomaly within the image.

2. The system of claim 1, wherein activating the first mucosa identification module is based on receiving endoscope type or model information identifying the endoscope.

3. The system of claim 2, wherein receiving endoscope type or model information includes receiving lighting information identifying the different lighting types the lighting component is capable of generating.

4. The system of claim 1, wherein the memory further includes instructions, which when executed by the processing circuitry, cause the processing circuitry to perform operations comprising:
continually monitoring images extracted from the image stream received from the endoscope using the first mucosa identification module; and
executing a second detection module from the first plurality of detection and characterization modules based on the first mucosa identification module identifying the mucosal tissue type as a second mucosal tissue type.

5. The system of claim 4, wherein the executing the second detection module includes outputting an indication to the display device that the endoscope has begun imaging a different region of the GI tract.

6. The system of claim 1, wherein the monitoring the image stream from the endoscope using the first mucosa identification module includes implementing a machine learning model, trained based at least in part on training data including a plurality of labeled mucosa images from various locations within a GI tract, to predict location of the endoscope within a GI tract based on real-time mucosal imaging from the endoscope.

7. The system of claim 1, wherein the monitoring the image stream using the first mucosa identification module includes periodically processing an image extracted from the image stream using the first mucosa identification module.

8. The system of claim 1, wherein the executing the first detection module includes selecting a first imaging modality from a plurality of imaging modalities supported by the endoscope.

9. The system of claim 8, wherein identifying the region of interest includes selecting a second imaging modality from the plurality of imaging modalities supported by the endoscope.

10. The system of claim 9, wherein the memory further includes instructions, that when executed by the processing circuitry, cause the processing circuitry to perform operations comprising executing a first characterization module from the plurality of detecting and characterization modules based at least in part on identifying the region of interest.

11. The system of claim 10, wherein executing the first characterization module includes outputting an indication of a tissue classification and corresponding confidence score related to the region of interest to the display device.

12. The system of claim 8, wherein identifying the region of interest includes displaying a user prompt on the display device providing an option to activate a second imaging modality from the plurality of imaging modalities supported by the endoscope.

13. A system, comprising:
one or more processing units; and
a computer-readable medium having encoded thereon computer-executable instructions to cause the one or more processing units to:
receive, during a medical examination, image data that is generated by a medical imaging device;
provide, at a first time during the medical examination, a first portion of the image data to one or more anatomy identification modules that is configured to output individual indications of whether individual portions of the image data depict a first anatomy type or a second anatomy type;
receive, from the one or more anatomy identification modules, a first indication that the first portion of the image data depicts the first anatomy type;
responsive to the first indication, deploying a first CAD model to analyze the first portion of the image data, wherein the first CAD model is configured to generate annotations in association with anomalies depicted in images of the first anatomy type;
provide, at a second time during the medical examination that is subsequent to the first time, a second portion of the image data to the one or more anatomy identification modules;
receive, from the one or more anatomy identification modules, a second indication that the second portion of the image data depicts the second anatomy type; and
responsive to the second indication, deploying a second CAD model to analyze the second portion of the image data, wherein the second CAD model is configured to generate annotations in association with anomalies depicted in images of the second anatomy type.

14. The system of claim 13, wherein the computer-executable instructions further cause the one or more processing units to:
receive an output from the first CAD model that indicates a detection of an anomaly within the first portion of the image data; and
responsive to the output from the first CAD model, deploying a third CAD model to analyze at least some of the first portion of the image data, wherein the third CAD model is configured to generate an output that classifies the anomaly detected by the first CAD model.

15. The system of claim 14, wherein deploying the third CAD model includes activating an alternative lighting modality on the medical imaging device.

16. The system of claim 15, wherein the alternative lighting modality is a narrow band imaging modality of blue or red light.

17. The system of claim 14, wherein the third CAD model is further configured to output a tissue classification and confidence score related to the tissue classification.

18. The system of claim 13, wherein the first anatomy type is squamous mucosa corresponding to an esophageal anatomical region and the second anatomy type is gastric mucosa corresponding to a stomach anatomical region.

19. The system of claim 13, wherein the image data comprises a sequence of multiple image frames that are generated during the medical examination by an endoscope.

20. The system of claim 13, wherein deploying the first CAD module includes outputting an indication of the first anatomy type.

21. The system of claim 13, wherein deploying the second CAD model includes outputting an indication of the second anatomy type.
